(19) United States
(12) Patent Application Publication
(10) Pub. No.: US 2003/0228338 A1
(43) Pub. Date: Dec. 11, 2003

(54) Label scent personal perfume applicator

(76) Inventor: Jaime Martinez, New York, NY (US)

Correspondence Address: Mark Manley, 5205 W 32nd, Sedalia, MO, 65301, US

(21) Appl. No.: 10/319,265

(22) Filed: Dec. 13, 2002

Related U.S. Application Data
(60) Provisional application No. 60/386,096, filed on Jun. 6, 2002.

(51) Int. Cl. A61K 6/00

(52) U.S. Cl. 424/401

(57) ABSTRACT

An adhesive label for applying deodorant, cologne or perfume. The label can be attached to the clothing or other personal items carried with a person such that the user never has to come in contact with the deodorant. The label can be useful to anyone but particularly to people who are allergic or sensitive to chemicals that might be in the deodorant. The label includes a pressure sensitive adhesive surface that will easily adhere to a variety of personal items including clothing, shoes or purses. The label can be easily removed from the surface by pulling up on one corner of the label to peel it off. The label provides an easy way to apply or remove a scent to a person.

### BACKGROUND

It is common practice to use cologne, perfume, deodorant or other aromatic products (herein after deodorant) by applying these compositions directly to the skin or clothing. Application can occur either as a spray on, roll on or as some other direct application. One problem with these applications is that they are relatively uncontrolled. Sprays can over-spray and once applied can not be removed until they are either washed off in a shower or until they wear off. Another problem is that some people are allergic to some chemicals in the deodorant applied. Deodorants applied directly to the skin or even to the clothing can cause an allergic reaction. If a person with sensitivity applies deodorant to the outer surface of clothing such as by spraying, there is a good chance that a portion of the deodorant will still pass through openings in the fabric weave of the clothing and still potentially cause problems.

Another issue is the health concern some people have about applying chemicals to their bodies. Particularly, there has been a concern that some people who regularly apply deodorants after shaving may suffer long term health problems as a result. Further, most conventional deodorants come in a can or bottle that is too large to carry on their person and are usually applied in the morning prior to leaving home. Even smaller containers meant for travel can spill or leak, and are typically still not small enough for ease of travel by any potential user.

There is a need for a deodorant applicator technique that does not require deodorant to be applied directly to the skin or sprayed on clothing. There is a need for a deodorant applicator that is small and easy to carry on ones person. There is also a need for a deodorant applicator that can not leak or spill even in situations where it is crushed.

### SUMMARY OF THE INVENTION

An object of the invention is to provide an easy way to apply deodorant without applying it directly to the skin or clothing of the user. Deodorant absorbed into a material such as open cell foam can be temporarily adhered to a variety of surfaces where it is unlikely to come into direct contact with the user.

A further object is to provide a label that has an adhesive backing that can be attached to personal articles that are near or on the person of the user but will not allow the deodorant containing label to come into direct contact with the skin of the user. The adhesive is of the pressure contact type that can be easily removed after use without damage to the surface the label was applied to.

A still further object of the present invention is to provide a deodorant applicator that is easy to carry and that can be used in any situation. The applicator is packaged for ease of use and can be disposed of when it is used up.

### DETAILED DESCRIPTION OF THE DRAWINGS

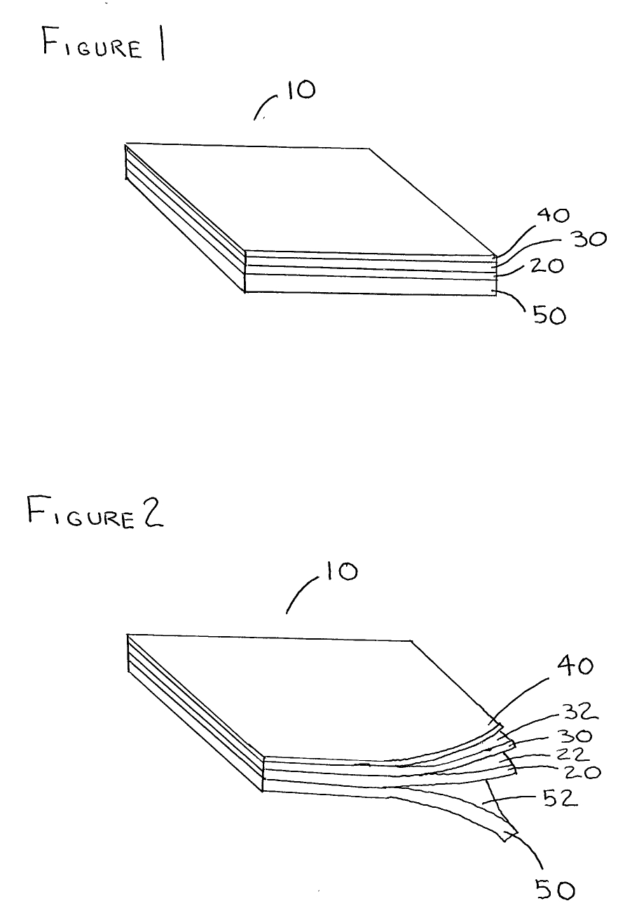

FIG. 1 shows a perspective view of the deodorant applicator label;

FIG. 2 shows a partially disassembled view of the deodorant applicator label;

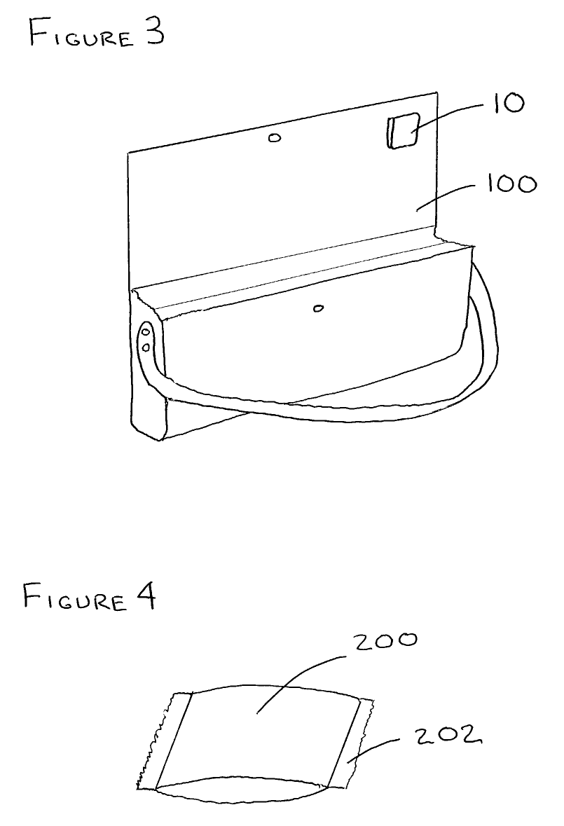

FIG. 3 shows a perspective view of the applicator label in use; and

FIG. 4 shows a perspective view of the applicator label in a package.

### DETAILED DESCRIPTION OF THE PREFERRED EMBODIMENT

FIG. 1 shows a perspective view of the deodorant applicator label 10. The device can consist of several layers, FIG. 1 exaggerates the layer thickness for clarity. The label 10 consists of a deodorant carrying layer 20. This deodorant layer 20 can be of an open cell material. Open cell foam or paper fiber can both be used for example. An adhesive layer 30 lays over the top of the open cell layer 20. The outer adhesive layer 30 can be a paper layer having adhesive applied to both sides. A protective layer 40 lays over layer 30 to protect one side of the adhesive. The protective layer can be of a material such as wax paper that can be separated from the adhesive easily by the user. Layer 50 is a decorative layer and in use can be the portion visible when the label 10 is in use.

FIG. 2 shows the layers of label 10 separated to more clearly show details of the label 10. Open cell layer 20 can include an adhesive side 22 to adhere it to layer 30. Outer paper layer 30 will also have an adhesive surface 32 that will be used when the label is to be applied. The adhesive surface 32 temporarily adheres the waxy protective layer 40 to the label 10. Decorative layer 50 is adhered to layer 20 using an adhesive layer 52.

FIG. 3 shows the deodorant label 10 in use applied to a personal item such as a purse 100. It will be clear that the label 10 could also be applied to clothing. When applied the label 10 can either be applied to a surface where it will not be seen or it can be applied to a surface where it can be seen. When applied to a visible surface a variety of colors, shapes or insignia can be printed on decorative surface 50. It should also be apparent that while the label 10 is shown square, a variety of decorative shapes can be used. The flexible label can be applied to clothing surfaces such as the tag often found in the collar of shirts. It can be applied to the front or back of this tag depending on if the user is concerned about coming into contact with the label.

FIG. 4 shows one packaging arrangement. In this case a single label 10 is packaged in a cellophane or similar package 200 which is sealed at each end 202. When the label 10 is needed the user can simply tear the cellophane package 200 open from either end 202. It will be apparent that while the label 10 has been shown individually wrapped, it could also be packaged in a variety of packaging arrangements. For example, the label 10 could be packaged as an individual sample that could allow a user to test a deodorant fragrance. The label 10 could be packaged in rolls with frangible edges between individual labels 10 that would allow them to be torn off the roll. The labels 10 could also be stacked in boxes similar to facial tissues, or packaged with multiple labels 10 loose in a box or bag. In use, the package 200 can be torn open and the individual label 10 removed. The user can then peel off the waxy protective layer 40 and dispose of it. The label can then be stuck to any surface using adhesive layer 32. The adhesive on layer 32 can be of the pressure application type that can be temporarily adhered to a surface and then later removed without damaging the surface used.

1. An adhesive label for use in applying deodorant comprising;
an adhesive layer including an adhesive surface,
an open cell layer, said open cell layer soaked in a deodorant,
a decorative surface applied to at least one surface of the open cell layer.

2. The adhesive label of claim 1 wherein the adhesive surface includes a coating of pressure sensitive adhesive.

3. The adhesive label of claim 1 wherein the open cell layer is made from open cell foam.

4. The adhesive label of claim 1 wherein the open cell layer is made from cellulose.

5. The adhesive label of claim 1 including a layer of waxy material temporarily placed over said adhesive surface.

6. The adhesive label of claim 1 wherein the adhesive surface comprises an adhesive easily removed by pulling on the label.

7. A combination adhesive deodorant label and personal item to be carried with a person comprising;
said adhesive deodorant label including an adhesive layer including an adhesive surface,
an open cell layer, said open cell layer containing a deodorant,
a decorative surface applied to at least one surface of the open cell layer wherein said adhesive deodorant label is applied to said personal item using the adhesive surface.

8. The combination of claim 7 wherein the adhesive layer includes a coating of pressure sensitive adhesive and wherein the open cell layer is made from open cell foam.

9. The combination of claim 7 wherein the open cell layer is made from cellulose fiber.

10. The method of applying deodorant including the steps of;
cutting an open cell material to a desired shape;
impregnating said open cell material with deodorant;
applying adhesive to at least one surface of said material;
temporarily covering said adhesive on the one surface with a layer of waxy paper;
removing said layer of waxy material and adhering said label to a personal item.
